(19) United States
(12) Patent Application Publication
(10) Pub. No.: US 2022/0186327 A1
(43) Pub. Date: Jun. 16, 2022

(54) SYSTEM FOR RAPID ON-SITE TESTING FOR AIRBORNE AND OTHER PATHOGENS

(71) Applicant: Inspirotec, Inc., North Chicago, IL (US)

(72) Inventors: Julian Gordon, Lake Bluff, IL (US); Rachel Reboulet, Chicago, IL (US); Osama Abdullah, Chicago, IL (US)

(21) Appl. No.: 17/552,288

(22) Filed: Dec. 15, 2021

Related U.S. Application Data
(60) Provisional application No. 63/126,036, filed on Dec. 16, 2020.

(51) Int. Cl. C12Q 1/70; B01L 3/00; C12Q 1/6806

(52) U.S. Cl. CPC C12Q 1/701; B01L 3/5023; C12Q 1/6806; B01L 2300/069; B01L 2300/042; B01L 2300/0832; B01L 2300/0848

(57) ABSTRACT

A simplified system for air sample collection and analysis for the presence of airborne pathogen is disclosed. An extraction device is used for extracting biomaterial previously deposited on the surface of a capture element of a sampling device, the capture element being of a select cross section and size. The extraction device comprises an upwardly opening head being wider at a top and narrower at a central opening at a bottom. An elongate cavity is attached to the enlarged head at the bottom extending downward from the head and closed at a bottom end to define an interior space, the cavity having a cross section corresponding to the cross section of the capture element and of a slightly larger size than the size of the capture element. An extraction fluid is in the cavity interior space. In use, insertion of the capture element through the head into the elongate cavity extrudes the extraction fluid liquid through the interface between the head and the elongate cavity and provides a fluidic shearing force that serves to solubilize biomaterial from the surface of the capture element.

### CROSS-REFERENCE TO RELATED APPLICATIONS

This application claims priority of Provisional Ser. No. 63/126,036, filed Dec. 16, 2020, the disclosure of which is hereby incorporated by reference in its entirety.

### FEDERALLY SPONSORED RESEARCH OR DEVELOPMENT

Not Applicable.

### MICROFICHE/COPYRIGHT REFERENCE

Not Applicable.

### FIELD OF THE INVENTION

This application relates to a simplified system for air sample collection and analysis for the presence of airborne pathogens

### BACKGROUND OF THE INVENTION

Severe acute respiratory syndrome coronavirus 2 (SARS-CoV-2) responsible for COVID-19 has rapidly spread across the globe since its likely origins in China during late 2019 (1). The dynamics of the COVID-19 pandemic have proven to be complex. There are many challenges pertaining to a better understanding of the epidemiology, pathology, and transmission of COVID-19. The clinical manifestations of COVID-19 range from an asymptomatic infection, mild respiratory illness to pneumonia, respiratory failure, multiorgan failure, and death (2). Transmission by surface contamination, fomites, fecal-oral routes, as well as airborne transmission have been proposed (3), and WHO recommendations focused on surface contact as major transmission. However, a group of leading researchers emphasized that the airborne route should not be neglected and is of key importance (4, 5). Control of the disease will necessitate tracking the airborne virus, but measuring airborne COVID-19 presents great technical difficulties.

### Publications on Airborne COVID-19

While actual knowledge of shedding of virus by asymptomatic carriers or during the course of the disease is critical to understanding and controlling it's spread, published work has been sparse. All published work has used the most sensitive possible RT-qPCR detection methodology.

Santarpia et al (6) studied 13 individuals evacuated from a cruise ship, the first significant outbreak in the US. For sampling They used a Sartorius Airport MD8 operated at 50 lpm for 15 min with gelatin filters. They found 63% of samples positive in patient rooms, 58% in hallways, with mean concentration 2.4 copies/liter of air in rooms, 2.5 in hallways.

Ong et al (7) investigated three patients in intensive care units. They used the same sampler Sartorius Airport MD8 at 15 lpm for 15 min or SKC Filter cassettes with PTFE filters, 5 lpm for 4 hrs. Samples were in the presence of patients in isolation rooms. All air samples were negative despite environmental surface contamination.

Jiang et al (8) used MAS-100 ECO at 100 lpm in isolation wards, but did not specify the duration of sampling. They used an Anderson-type sampler and found one positive out of 27 locations.

Guo et al (9) investigated intensive care units and general wards using an SASS 2300 Wetted Wall Cyclone Sampler (Research International, Inc.) at 300 L/min for 30 min. They found positives in 12-44% of tested locations in a known contaminated area and found 0.5-3.8 copies/L of air.

Ding et al (3) used four different bioaerosol samplers in hospital isolation rooms: an Andersen one-stage viable impactor (QuickTake-30, SKC, USA; sampled at 10 L/min for 30 min), an AirPort MD8 (Sartorius, Germany; 50 L/min for 20 min), an ASE-100 (Langsi Medical Technology, Shenzhen, China; 500 L/min for 2 min, and 500 L/min for 20 min), and a WA-15 (Dinglan Technology, Beijing, China; sampled at 14 L/min for 30 min). A variety of collection media were used. Forty five of 46 samples were negative. The one positive sample was from the ASE-100 with a 20 minutes sampling time.

Binder et al (10) used NIOSH samplers at 3.5 lpm for 4 hrs. They were placed 1-3 m. from hospitalized patients. Out of 20 patient rooms, 3 rooms gave 1 positive air sample, and patient contact tracing yielded no significant positives.

Lednicky et al (11, 12) used an air sampling method VIVAS (13) aimed at optimizing viable virus capture, at 6.5 lpm for 1 hour. In a student health center, they found SARS-CoV-2 virus by the most sensitive method of Sanger sequencing, but failed to identify viable virus as they were outgrown by 2 other coronaviruses and influenza virus. They suggest longer sampling times might be required (11). In a study in a hospital room with two COVID-19 patients in 4 samples taken within 2-4.8 m from patients, they found 16-94 genome equivalents/L of air (12). They also cultured samples and showed the presence of viable virus. This is the only work showing viable virus. All other work on air samples was analysis for viral RNA by PCR methodology.

The foregoing summary of published works illustrates the challenge of routinely measuring airborne COVID-19 due to lack of standardized methodology, trial and error, use of different samplers, and different sampler times.

### Publications on Simplification of Detection Technology

In parallel with the foregoing work on airborne methods, the need to increase the ease of COVID-19 has been paralleled by work on simplification of diagnostic methods to make it more widely available and possibly performable outside of controlled laboratory environments. Loop mediated amplification (LAMP) originally established by Eiken (U.S. Pat. Nos. 9,909,168; 7,374,913; 7,851,186; 7,846,695) has been adapted to COVID-19 testing by several groups. New England Biolabs provides a primer tool for designing the 6 primers for target genes (https://lamp.neb.com/#!/). The isothermal amplification typically requires an incubation time of 30 minutes at 65° C. At the end of that time, sufficient nucleoside triphosphate substrate will have been digested in the polymerization. Amplification can be detected by precipitation of magnesium pyrophosphate, observed as turbidity and pH change resulting in color shift (U.S. Pat. Nos. 10,253,357 and 10,724,079), fluorescent intercalating dyes, or liquid crystal violet (14, 15) showing ds DNA product. Result can be scored with an appropriate instrument or observed visually.

Kits of master mix for LAMP are available from a variety of commercial sources but are designed to work with purified RNA. RNA purification usually involves laboratory procedures that require skills, and/or equipment that do not readily themselves to field use.

Klein et al (16) used a novel magnetic bead method to isolate COVID-19 RNA for amplification by LAMP. Upper respiratory tract samples in transport medium were lysed with 5M guanidinium thiocyanate, 40 mM dithiothreitol, 20 ug/ml glycogen, 1% Triton x-100, 25 mM sodium citrate, pH 8, placed in 96 well plates, mixed with magnetic beads, washed 3 times with ethanol, and eluted with water.

Thi et al (17) attempted to bypass separate RNA preparation by transferring the sample from transport medium directly to LAMP master mix, heating for 5 minutes at 95°, then transferring to a second plate with LAMP master mix and heating for 30 minutes at 65°. This was sufficient to detect COVID-19 RNA in high titer samples.

Yu et al (18) say that LAMP is less demanding of RNA purification than RT-qPCR but nevertheless used purified RNA samples. They used a novel fluorescent dye (GeneFinder D039 from Bridgen, http://www.bridgen.cn/index.php?m=content&c=index&a=show&catid=17&id=367) which fluoresces green in blue light.

Zhang et al (19) heat inactivated a swab sample in transport medium for 30 minutes at 56° and performed RNA extraction with an automated system. Six out of seven patient samples were positive by both LAMP and RT-qPCR; the 7th patient sample was negative by both methods. They also tested samples that were spiked into HeLa cell lysate as evidence that the RNA preparation step could be obviated. However, they did not attempt to do LAMP on the clinical samples. The same group showed guanidine hydrochloride in the master mix improved the COVID-19 (20), but testing was with synthetic RNA so they do not suggest this as a way of simplifying the protocol.

Kellner et al described an “open access assay” (21). with robustness, compatibility with crude patient samples, enhanced sensitivity, compatibility with home testing setups, and avoiding access to the “patent protected gold-standard RT-LAMP enzymes”. They used a first lysis step then a colorimetric reaction with hydroxynapthol blue as this provided increased sensitivity and made the final reaction mix less sensitive to pH changes introduced by sample type and collection method. The color reaction depends on the reduction in free Mg++ in the medium as it forms complexes with pyrophosphate released from the LAMP substrates. For enhanced sensitivity, they proposed use of magnetic beads to enrich the RNA from lysates. The do not suggest omission of the lysis as a separate step. They describe an alternative that passes RNA purification as a separate step but use RNA concentration on magnetic beads.

Butler et al (22) did extensive comparison of LAMP with RT-qPCR and showed extensive concordance. All work was done with purified RNA from a variety of sources. They do not consider adding unpurified samples to LAMP mix.

Anahtar et al (23) describe a 40 min from sample to result LAMP assay by direct addition of the sample to the standard LAMP assay, but the sample volume that could be added was severely limited, resulting in very low sensitivity, >300 gene copies per sample.

Rabe et al (24) describe a simple inactivation and purification protocol that avoids the use of commercial kits for RNA purification. They describe a method for inactivation of virus and inhibition of RNase, including heating at 95° C. for 5 minutes. Their glass milk RNA purification step improved the limit of detection by a factor of 10. Due to the lack of sensitivity, negative samples need to be re-run by a reference lab. Nowhere do they suggest a procedure where there are not separate inactivation and RNA purification steps.

All of the above methods involve collection of the human sample in some transport medium or other. Transport media have been developed and used over the years to maximize the stability of a variety of pathogens in transport from patient to laboratory. Swabs have been known since 1893. Amies media has been used since 1967 (25). Other transport media are described in the above prior art. The need to stabilize the transported virus is therefore widely considered as crucial. However, none anticipates the use of pure water as an extraction medium which can be used immediately on collection in an off-site location for stabilization of sample if repeat testing is required.

Carter et al (26) were able to add, SybrSafe dye (Life Technologies,(Z)-4-((3-Methylbenzo[d]thiazol-2(3H)-ylidene)methyl)-1-propylquinolin-1-ium 4-methylbenzenesulfonate) directly into the LAMP master mix, in assays for Zika virus, instead of SYBR Green which inhibits the reaction. They as well as Chen et al .(27), in an assay for Coxiella burnetiid, showed that master mix can be lyophilized, but maintained that SYBR Green can be included with lyophilized Master mix.

### Publications

1. F. He, Y. Deng, W. Li, Coronavirus disease 2019: What we know? Journal of Medical Virology 92, 719-725 (2020).
2. W.-j. Guan et al., Comorbidity and its impact on 1590 patients with COVID-19 in China: a nationwide analysis. European Respiratory Journal 55, (2020).
3. Z. Ding et al., Toilets dominate environmental detection of severe acute respiratory syndrome coronavirus 2 in a hospital. The Science of the total environment 753, 141710-141710 (2020).
4. L. Morawska et al., How can airborne transmission of COVID-19 indoors be minimised? Environment International 142, (2020).
5. L. Morawska, D. K. Milton, It is Time to Address Airborne Transmission of COVID-19. Clinical infectious diseases: an official publication of the Infectious Diseases Society of America, (2020).
6. J. L. Santarpia et al., Aerosol and surface contamination of SARS-CoV-2 observed in quarantine and isolation care. Scientific Reports 10, (2020).
7. S. W. X. Ong et al., Air, Surface Environmental, and Personal Protective Equipment Contamination by Severe Acute Respiratory Syndrome Coronavirus 2 (SARS-CoV-2) From a Symptomatic Patient. Jama-Journal of the American Medical Association 323, 1610-1612 (2020).
8. Y. Jiang et al., Clinical Data on Hospital Environmental 1 Hygiene Monitoring and Medical Staff Protection during the Coronavirus Disease 2019 Outbreak. medRxiv preprint doi: https://doi.org/10.1101/2020.02.25.20028043, (2020).
9. Z. D. Guo et al., Aerosol and Surface Distribution of Severe Acute Respiratory Syndrome Coronavirus 2 in Hospital Wards, Wuhan, China, 2020. Emerging Infectious Diseases 26, 1586-1591 (2020).
10. R. A. Binder et al., Environmental and Aerosolized Severe Acute Respiratory Syndrome Coronavirus 2 Among Hospitalized Coronavirus Disease 2019 Patients. The Journal of infectious diseases 222, 1798-1806 (2020).
11. J. A. Lednicky et al., Collection of SARS-CoV-2 Virus from the Air of a Clinic within a University Student Health Care Center and Analyses of the Viral Genomic Sequence. Aerosol and Air Quality Research 20, 1167-1171 (2020).
12. J. A. Lednicky et al., Viable SARS-CoV-2 in the air of a hospital room with COVID-19 patients. International journal of infectious diseases: IJID: official publication of the International Society for Infectious Diseases 100, 476-482 (2020).
13. M. Pan et al., Efficient collection of viable virus aerosol through laminar-flow, water-based condensational particle growth. Journal of Applied Microbiology 120, 805-815 (2016).
14. M. El-Tholoth, H. H. Bau, J. Song, A Single and Two-Stage, Closed-Tube, Molecular Test for the 2019 Novel Coronavirus (COVID-19) at Home, Clinic, and Points of Entry. ChemRxiv : the preprint server for chemistry, (2020).
15. S. Miyamoto, S. Sano, K. Takahashi, T. Jikihara, Method for colorimetric detection of double-stranded nucleic acid using leuco triphenylmethane dyes. Analytical Biochemistry 473, 28-33 (2015).
16. S. Klein et al., SARS-CoV-2 RNA Extraction Using Magnetic Beads for Rapid Large-Scale Testing by RT-qPCR and RT-LAMP. Viruses-Basel 12, (2020).
17. V. L. D. Thi et al., A colorimetric RT-LAMP assay and LAMP-sequencing for detecting SARS-CoV-2 RNA in clinical samples. Science Translational Medicine 12, (2020).
18. L. Yu et al., Rapid Detection of COVID-19 Coronavirus Using a Reverse Transcriptional Loop-Mediated Isothermal Amplification (RT-LAMP) Diagnostic Platform. Clinical Chemistry 66, 975-977 (2020).
19. Y. H. Zhang et al., Rapid Molecular Detection of SARS-CoV-2 (COVID-19) Virus RNA Using Colorimetric LAMP. medRxiv preprint doi: https://doi. org/10.1101/2020.02.26.20028373, (2020).
20. Y. Zhang et al., Enhancing colorimetric loop-mediated isothermal amplification speed and sensitivity with guanidine chloride. BioTechniques 69, 178-185 (2020).
21. Kellner M et al., A rapid, highly sensitive and open-access SARS-CoV-2 detection assay for laboratory and home testing. https://doi.org/10.1101/2020.06.23.166397, (2020).
22. D. Butler et al., Host, Viral, and Environmental Transcriptome Profiles of the Severe Acute Respiratory Syndrome Coronavirus 2 (SARS-CoV-2). https://doi. org/10.1101/2020.04.20.048066, (2020).
23. M. Anahtar et al., Clinical assessment and validation of a rapid and sensitive SARS-CoV-2 test using reversetranscription loop-mediated isothermal amplification. https://doi.org/10.1101/2020.05.12.20095638, (2020).
24. B. A. Rabe, C. Cepko, SARS-CoV-2 detection using isothermal amplification and a rapid, inexpensive protocol for sample inactivation and purification. Proceedings of the National Academy of Sciences of the United States of America 117, 24450-24458 (2020).
25. C. R. Amies, MODIFIED FORMULA FOR PREPARATION OF STUARTS TRANSPORT MEDIUM. Canadian Journal of Public Health 58, 296-300 (1967).
26. C. Carter, K. Akrami, D. Hall, D. Smith, E. Aronoff-Spencer, Lyophilized visually readable loop-mediated isothermal reverse transcriptase nucleic acid amplification test for detection Ebola Zaire RNA. Journal of Virological Methods 244, 32-38 (2017).
27. H.-W. Chen, W.-M. Ching, Evaluation of the stability of lyophilized loop-mediated isothermal amplification reagents for the detection of Coxiella burnetii. Heliyon 3, (2017).

A new device for human testing for COVID-19 from the company Lucira (https://www.lucirahealth.com/) has recently received FDA emergency approval. From the FDA approval letter accessible online:

“Your product is a rapid, single-use molecular diagnostic test kit for the qualitative detection of SARS-CoV-2 RNA from self-collected nasal swab specimens that contains all the components required to perform testing. To use your product, SARS-CoV-2 nucleic acid is first eluted and lysed from nasal swabs that are inserted into the Sample Vial. The eluant then enters into the Test Unit where the nucleic acid is then reverse transcribed (RT) into cDNA followed by loop-mediated isothermal amplification (LAMP) and detected by the Test Unit as a color change. Test results are displayed on the Test Unit via LED indicators. The Lucira COVID-19 All-In-One Test Kit includes the following materials or other authorized materials: Sterile Nasal Swab, Sample Vial, Test Unit, Batteries, Plastic Disposal Bag.”

From this it can be deduced that they have combined extraction/transport fluid with lysis medium to release RNA in the first step. Thereafter the steps take place internally in the device and there is not suggestion that RNA purification has been eliminated as a separate step carried out automatically inside their closed box. Further, the description implies that reverse transcription and LAMP are performed as separate steps.

This application relates to improvements in air sample collection and analysis for the presence of airborne pathogens.

### SUMMARY OF THE INVENTION

In the current pandemic the invention described herein fulfills the great need for high-volume low-cost tests that can be performed rapidly at the suspicious site by unskilled workers so that immediate avoidance or remediation can be performed. The invention comprises a system wherein the sample is collected as an air sample with a low cost easily deployable air sampling device, efficiently and seamlessly transferred from a collection device to an extraction device. The extraction device is a simple funnel-like device that requires only the insertion of the sample collector into a cavity, displacing the sample into a location where it can be collected in a transfer pipette in transport medium with a minimum of dilution, to the Master Mix where a LAMP amplification takes place, permitting the visual reading of a result within 30 minutes at 65° C. There are no further manipulations involved which increase skill requirements and introduce the risk of user error, as do all of the prior art protocols summarized in the publications section.

It will be apparent to one skilled in the art that the disclosed invention can be applied to a multiplicity of pathogens and sampling methods. Other viruses such as influenza viruses, cold viruses or other corona viruses and influenza viruses may be analyzed by LAMP or similar isothermal amplification methods. This is especially important as other viruses may present symptoms similar to COVID-19. Other sampling methods may be used, such as for human samples or environmental surface samples. Human samples may be nasopharyngeal or saliva or any other relevant bodily fluid. Samples can be collected on swabs as well as the electrodes of the air sampler described in detail in this document. Funnel-like devices can be designed for swabs or any other collection means, to permit efficient and reproducible transfer of sample to transport medium. This is also safer, since the transfer takes place in a closed system, not in the open tubes conventionally used, and excess sample can be safely disposed of in the same closed vessel. It is a surprising finding of the disclosed invention that pure RNase free water may be used as the transport medium and sample may be stable in it if needed for repeat assays. A further aspect of the invention is the surprising finding that the introduction of a suitable amount of detergent to known master mixes permits the lysis of virus and release of amplifiable RNA within the mix, so that no separate inactivation or RNA purification step is required. Together the advances result in a simple one step procedure that may be performed by an unskilled operator and requiring only a transfer pipette and, a fixed temperature heating block to provide the 30 minutes at 65° C.

In accordance with one aspect of the invention, there is disclosed an extraction device for extracting biomaterial previously deposited on the surface of a capture element of a sampling device, the capture element being of a select cross section and size. The extraction device comprises an upwardly opening head being wider at a top and narrower at a central opening at a bottom. An elongate cavity is attached to the enlarged head at the bottom extending downward from the head and closed at a bottom end to define an interior space, the cavity having a cross section corresponding to the cross section of the capture element and of a slightly larger size than the size of the capture element. An extraction fluid is in the cavity interior space. In use, insertion of the capture element through the head into the elongate cavity extrudes the extraction fluid liquid through the interface between the head and the elongate cavity and provides a fluidic shearing force that serves to solubilize biomaterial from the surface of the capture element.

There is disclosed in accordance with another aspect, a method for extracting biomaterial previously deposited on the surface of a capture element of a sampling device, the capture element being of a select cross section and size. The method comprises providing an extraction device comprising an upwardly opening head being wider at a top and narrower at a central opening at a bottom, and an elongate cavity attached to the enlarged head at the bottom extending downward from the head and closed at a bottom end to define an interior space, the cavity having a cross section corresponding to the cross section of the capture element and of a slightly larger size than the size of the capture element. The cavity interior space is filled with an extraction fluid. The capture element is inserted through the head into the elongate cavity thereby extruding the extraction fluid liquid through the interface between the head and the elongate cavity and thereby providing a fluidic shearing force that serves to solubilize biomaterial from the surface of the capture element.

Other objects, features, and advantages of the invention will become apparent from a review of the entire specification, including the appended claims and drawings.

### BRIEF DESCRIPTION OF THE DRAWINGS

FIG. 1 is a top view of a funnel-like extraction device;

FIG. 2 is a perspective view of the device of FIG. 1;

FIG. 3A is a section taken along the line 3A-3A of FIG. 1 together with a capture electrode from an air sampler capture device;

FIG. 3B is a view similar to FIG. 3A with the capture electrode inserted in the extraction device;

FIG. 4A is a section taken along the line 4A-4A of FIG. 1 together with the capture electrode from an air sampler capture device;

FIG. 4B is a view similar to FIG. 4A with the capture electrode inserted in the extraction device;

FIG. 5A is a view similar to FIG. 3A with a transport fluid in the extraction device;

FIG. 5B is a view similar to FIG. 5A showing displacement of the transport fluid by insertion of the capture device electrode;

FIGS. 6A, 6B and 6C are exemplary engineering drawings showing the extraction device molded in two parts; and

FIG. 7 is a block diagram illustrating the steps in an exemplary LAMP procedure and including the simplification introduced in the disclosed invention.

### DETAILED DESCRIPTION OF THE PREFERRED EMBODIMENT

A simplified system for air sample collection and analysis for the presence of airborne pathogen is disclosed. The methodology is sufficiently versatile that it may also be adapted for use with human test samples as well as for samples collected from the air. Due to the urgency of the current pandemic, Applicant's patented technology for capture of airborne biomaterial utilizing electrokinetic propulsion is here used to capture biomaterial on stainless steel electrodes. A novel simplified method of collection of samples from the electrodes into an extraction/transport medium is shown. The electrode is simply pushed into the device pre-loaded with the medium where the electrode has limited clearance from the walls of the device and the shearing force of the liquid being forced though this limited clearance results in efficient collection. A similar device for human samples collected on a swab is described, where there is limited clearance between the swab and the vessel walls causes the extrusion of the extraction/collection fluid through the swab to effect efficient collection in one stroke, Further, advances in simplification of the method of reverse transcriptase loop-mediated isothermal amplification (RT-LAMP) are described. Addition of non-ionic detergent helps promote viral lysis and release of RNA without a heating step, and allows direct addition of the lysate to the RT-LAMP reaction mixture. The combination of simple and effective process steps results in an entire process that requires no skilled operator.

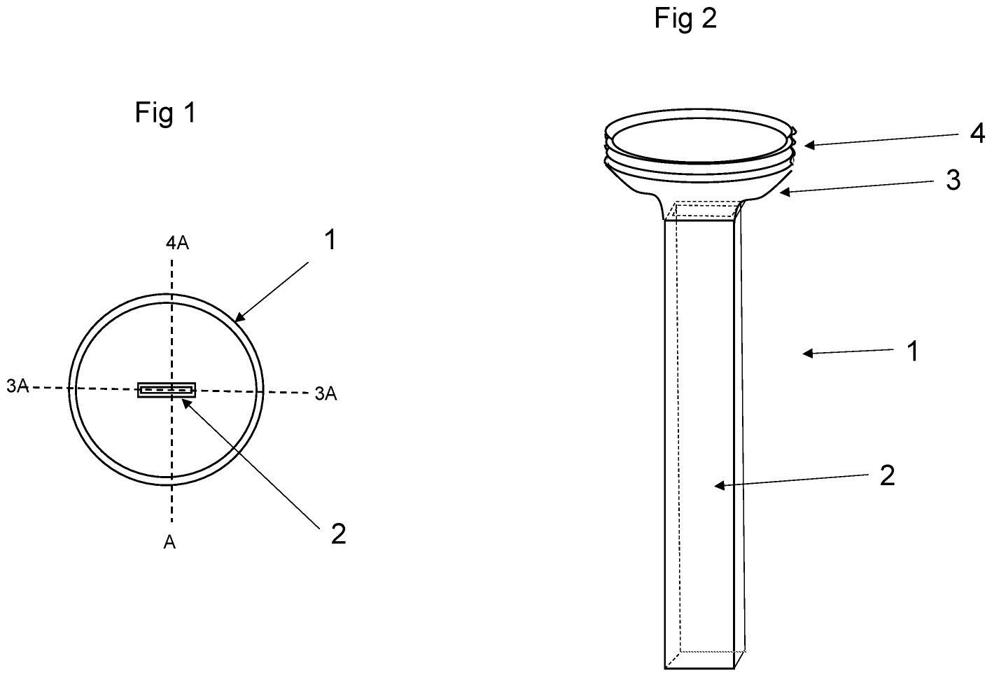

FIG. 1 shows top view of a funnel or pestle-like extraction device 1 in accordance with the invention. FIG. 2 is a perspective representation of the extraction device 1 of FIG. 1. This and subsequent figures are not drawn to any specific scale but are meant to illustrate the operating principles of the device, and appropriate scaling may be used for specific devices.

The extraction device 1 comprises an upwardly opening head 3 in the shape of a bowl. The head 3 is wider at a top end and narrow at a central bottom end which is open. The head 3 opens into an elongate cavity 2. The cavity 2 is of parallelepiped construction closed at a bottom end to define an interior space for receiving an extraction fluid and a capture element, such as an electrode, as described below. The cavity 2 is of rectangular cross section. The head 3 is open at an interface where it is connected to the cavity 2 for the collection of fluid displaced from the cavity 2 by the electrode as will become clear in the following figures. The head 3 includes a threaded outer wall 4 to removably receive a screw cap 8, discussed below, for sealing the extraction device with transport fluid in the cavity 2 for shipping and re-sealing for safe disposal. The invention is not limited to a threaded cap, but may also be a press cap or a stopper according to the desired specifications. The cap may include a gasket or the like to seal the device for transport.

The shape of the cavity is designed to conform to the shape of the capture element. If the capture element is cylindrical, then the cavity 2 will have a cylindrical interior cross section.

The extraction device 1 is advantageously formed of plastic. It could be formed in one piece by injection molding, blow molding or dip molding. Alternatively, the extraction device could be molded in multiple parts which are joined by ultrasonic welding or using an adhesive.

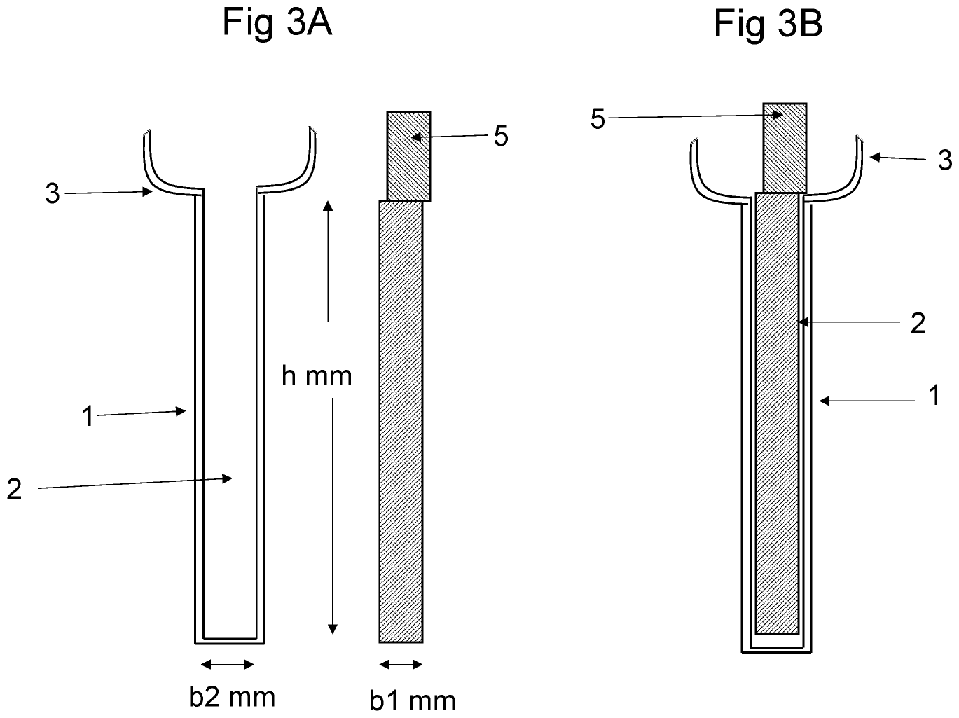

FIG. 3A shows a capture element, in the form of an electrode 5 from an air sampling device, positioned to one side of the extraction device 1. The electrode 5 is in the form of a flat strip, with a double reverse bend at one end and may be formed of metal, as necessary or desired. FIG. 3B shows the electrode 5 inserted in the cavity 2 of the capture device 1.

The illustrated electrode 5 is of a type shown in Applicant's ionic capture device, commercialized as AirAnswers® and described in U.S. Pat. No. 9,360,402, the specification of which is incorporated by reference herein. The ionic capture device captures airborne biomaterial utilizing electrokinetic propulsion on stainless steel electrodes. The electrodes are removeable for extraction of the captured biomaterial. As is apparent, the shape of the electrode shown in the drawings is by way of example only. Other shapes and materials may be used.

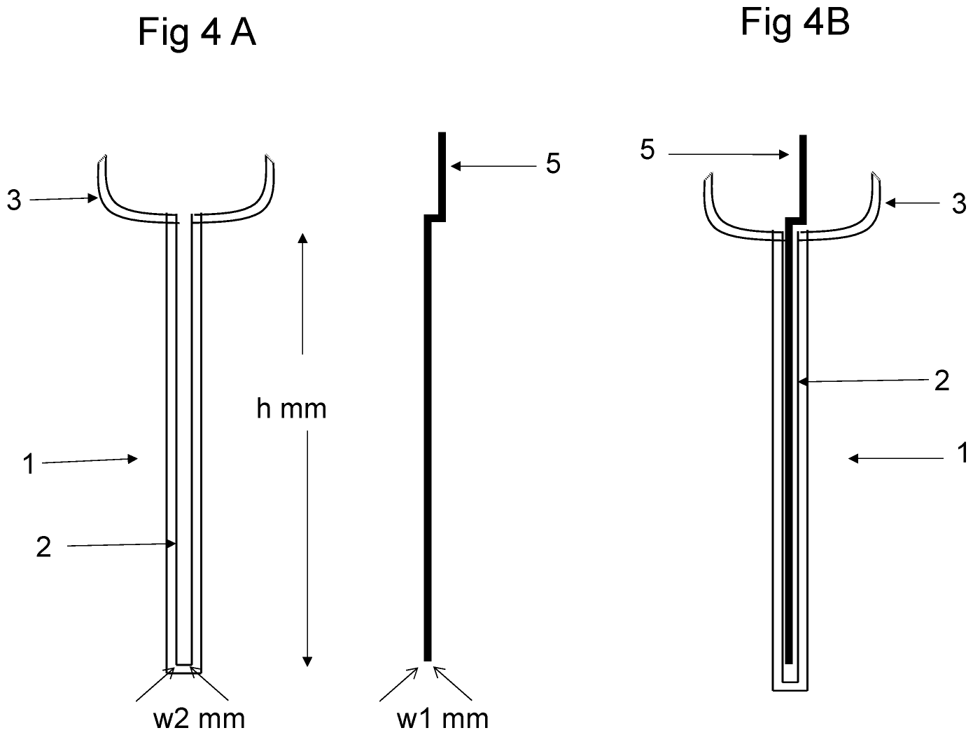

FIG. 3A shows a dimension b1, the width of the electrode 5, and b2, the width of the interior of the cavity 2. The length of the cavity is illustrated by the dimension h. Similar to FIGS. 3A and 3B, FIGS. 4A and 4B show side sections of the capture device 1 and the electrode 5, and show the dimensions w1, the thickness of the electrode 5 and the corresponding interior dimension w2 of the cavity 2.

Advantageously, the overall clearance between the electrode 5 and the walls of the cavity 2 should be minimized, but not be so tight that the electrode 5 will jam on insertion. The clearance between the capture element 5 and the interior walls of the cavity 2 should be less than about 1 mm, and advantageously less than 0.1 mm. As will be apparent, the minimization of this clearance is critical for the sample recovery in an extraction process.

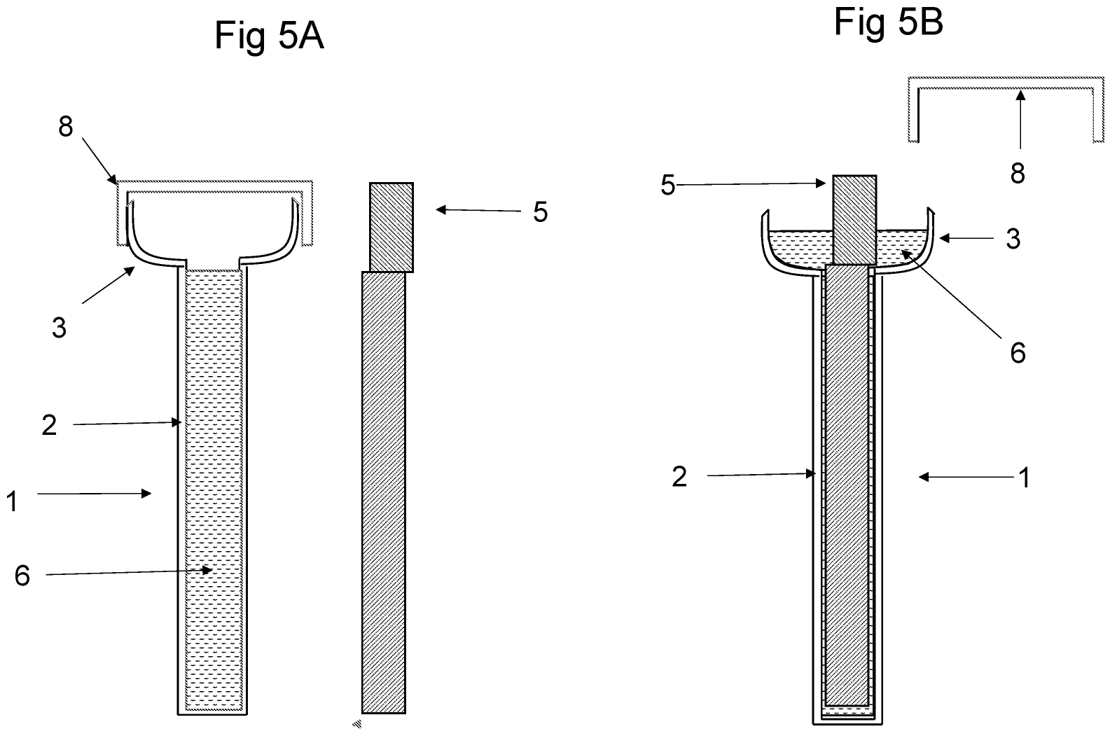

FIGS. 5A and 5B are similar to FIGS. 3A and 3B, respectively, but with the addition of a transport fluid 6 filling the cavity 2 as shown in FIG. 5A. The cap 8 is also shown secured to the head 3, such as by threading, which may be used to seal the device for shipping and will be removed for insertion of the electrode 5.

FIG. 5A shows the extraction device 1 before removal of the cap 8 and insertion of the electrode 5. FIG. 5B shows at 7 in the head 3 how the transport fluid 6 is displaced into the bowl-shaped head 3. Upon later removal of the cap 8, a sample may be withdrawn with a micropipette dispenser and added to a tube containing LAMP reaction mix for the detection reaction.

A is apparent, the clearance between the walls of the cavity 2 and the electrode 5 determine both the magnitude of a shearing effect when the electrode 5 is inserted into the cavity 2, thus ensuring efficient washing of the surface and solubilization of the captured bio-material, and will also maximize the volume of sample that will be recovered. The volume of fluid displaced by the electrode 5 is h*b2*w2−h*b1*w1. An example of this volume calculated from typical dimensions is shown in Table 1.

TABLE 1

| | h | w1 | b1 | w2 | b2 |
|---|---|---|---|---|---|
| Dimension | 80.0 | 0.6 | 5.0 | 0.7 | 5.1 |
| Electrode volume | 240.0 | | | | |
| Cavity volume | 285.6 | | | | |
| % displaced | 84.0 | | | | |

All dimensions are in mm, volumes in μL.

For the case illustrated, using a clearance of 0.1 mm, 84% of sample volume is captured in the head 3 in 240 μL.

It is apparent that the same principles described herein can be applied to a plurality of possible sample collection devices. Thus, for swab samples collected nasopharyngeal for patients or swabs used for environmental surface sampling, a circular cross-section device will be used, with the diameter of the circular section cavity designed to minimize the clearance between the swab and the walls of the cavity to provide the described shearing effect.

In all cases, the capture of sample into the transport medium may be preferably achieved by one thrust of the sample capture element 5 into the cavity 2. If it is desired to increase efficiency of capture into the transport medium, this may be repeated multiple times. Regardless, repeated insertion into the cavity 2 will give a more objective and reproducible capture than is achieved by current methods of swirling or vortex mixing.

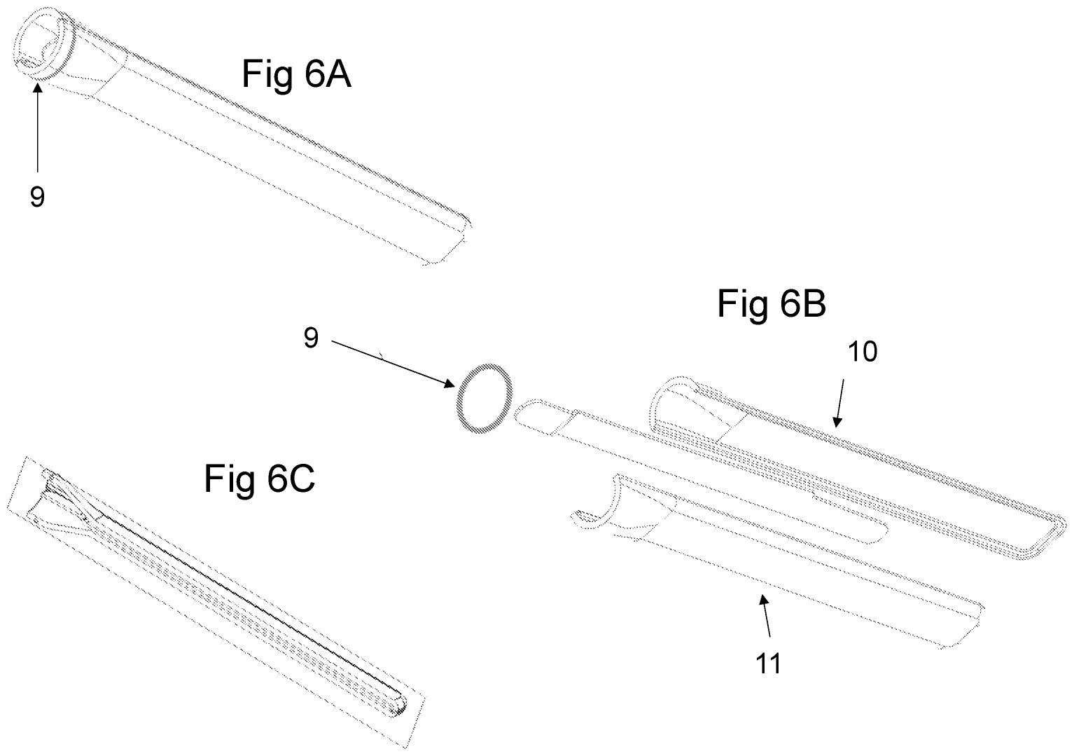

FIGS. 6A, 6B and 6C are created from a Solidworks™ design and show a perspective view in FIG. 6A, an exploded view in FIG. 6B and a cross section in FIG. 6C for an extraction device. This embodiment includes an O-ring shown at 9 in FIGS. 6A and 6B, to enable the separation of two halves 10 and 11, see FIG. 6B, of the device that would advantageously be molded as separate parts. The two halves could be secured by an adhesive or ultrasonic welding. The O-ring 9 provides a seal for a removable cap.

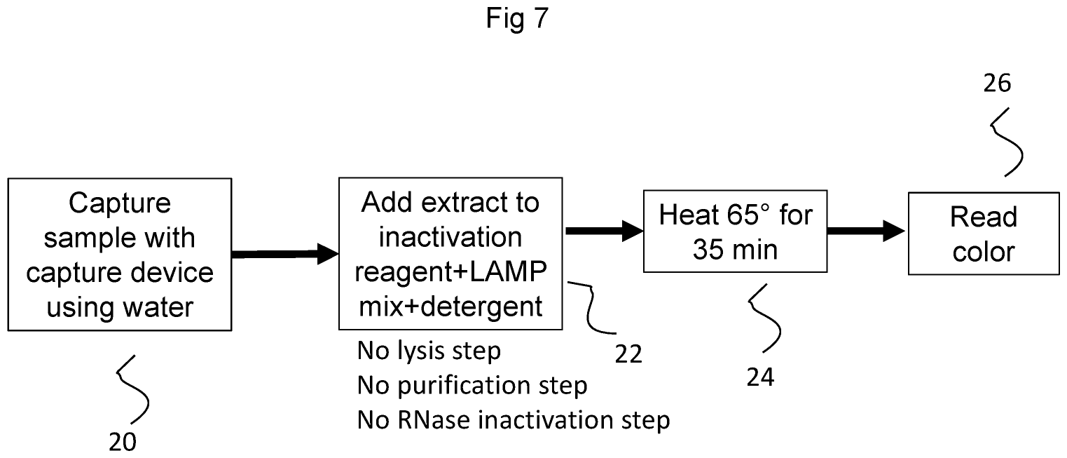

FIG. 7 is a block diagram illustrating the steps involved in the shows the steps involved in the sample collection in accordance with the invention. As described above, the prior LAMP systems used a heat inactivation step, RNA purification, concentration, and transfer to a master mix for the LAMP reaction. In the disclosed invention, all these steps are reduced to a single liquid transfer from the extractor to the LAMP master mix supplemented with other reagents as will be detailed in the example. In accordance with the invention, a sample is captured on the electrode 5 at a block 18, such as by using the described electrokinetic device. The electrode 5 is removed from the capture device and the sample from the electrode 5 is extracted with water at a step 20. The extraction is added to an inactivation reagent and LAMP mix and detergent at a block 22. There is no lysis step, no purification step, or RNase inactivation step. This mixture is then heated at 65° for 35 minutes at a block 24. The resultant color is read at a block 26.

### Example

The electrodes that have samples positive and negative for COVID-19 are extracted with pure RNase free water and an aliquot of that is added to the modified LAMP reaction mix as follows.

Inactivation reagent (IR):

0.358 g of Tris(2-carboxyethyl)phosphine hydrochloride (TCEP) in 2.5 mL nuclease free water, 2.2 mL of 1.1 N NaOH, and 1 mL of 0.5 EDTA).

1:10 Inactivation reagent: 90 μl nuclease free water, 10 μl Inactivation reagent.

Inactivation solution: Nuclease free water, 1:10 inactivation reagent, 0.1 Tween 20. (100:1:2) ratio for electrode samples and (100:1:1) ratio for nasopharyngeal samples.

LAMP Primers (from Integrated DNA Technologies, Coralville, Iowa): Gene N-A FIP 1.6 μM, Gene N-A BIP 1.6 μM, Gene N-A F3 0.2 μM, Gene N-A B3 0.2 μM, Gene N-A LF 0.4 μM, Gene N-A LB 0.4 μM.

LAMP primers stock: Gene N-A FIP 16 μL, Gene N-A BIP 16 μL, Gene N-A F3 2 μL, Gene N-A B3 2 μL, Gene N-A LF 4 μL, Gene N-A LB 4 μL, filled up to a 100 μl with Nuclease free water.

Reaction mix: 5 μl master mix (WarmStart® colorimetric LAMP 2X Master Mix, New England Biolabs, Ipswich, Mass.) and 3 μl of primers.

Pre-mix 10 ul sample with 1 ul inactivation solution by pipetting up and down the pipette together and add this to reaction mix tube.

Place sample in preheated heating block for 35 minutes at 65° C. and inspect color change. A typical result is shown in Table 2.

TABLE 2

| Sample | Result |
|---|---|
| Human Positive Sample | Orangish |
| Human negative sample | Pink |
| S17 - Cartridge positive sample | Yellow |
| S33 - Cartridge negative sample | Pink |
| S41 - Cartridge negative sample | Pink |
| Positive Control | Yellow |
| Negative Control | Pink |

Note that all positive samples, human and cartridge had Ct 25 for the human positive sample and 24 for the cartridge positive sample by conventional PCR. It will be apparent to one skilled in the art that the above procedure can be rapidly performed with multiple samples in parallel. Because of availability of positive samples, the above was performed on 10 μl. By lyophilization of master mix and primers, sample volume may be increased to 18 μl with corresponding increase in sensitivity. Methods using lyophilized reagents are described in the prior art section. The simplification of the procedure described, with omission of RNA purification and omitting a separate heat inactivation/lysis step, means that for the user, all that is required is dispensing sample into a tube containing inactivation solution and immediate transfer of that to the tube containing master mix and primers, then placing this tube in a heating block.

This simplified protocol resulted from the addition of Tween® 20 to the inactivation solution. This is not known from any known prior art. The Tween® may be replaced by any other of the non-ionic detergent, comprising the Tween®, Triton, and the Brij series. Unlike prior art systems, here we show that the presence of detergent and sulfhydryl reducing agent permits the viral lysis and RNA release, as is the presence of TCEP. The TCEP may be replaced by other sulphydryl reducing compounds that will break viral S—S bonds, such as di-thiothreitol or β-mercaptoethanol. Stability of such solutions may be improved by removal of dissolved oxygen from all buffers or water in which they will be dissolved. The method is also compatible with different variant transport media. The human samples here had been collected by conventional nasopharyngeal swap in buffered saline, or pure water for the samples from the samples collected from the air on stainless steel electrodes. An alternative would be to include ionic detergent in the transport/extraction medium for the electrode such that the final concentration in the mixture with inactivation solution will approximate that in the example. The detergent level may not be so high as to inhibit the final LAMP reaction. Limits to possible detergent concentrations are described in the prior art section.

There are thus innumerable variations and possible improvements that will be apparent to one skilled in the art within the scope of the invention.

The following sequences are used in this document:

SEQ ID NO: 1
Gene N-A FIP/ TCTGGCCCAGTTCCTAGGTAGTCCAGACGAATTCGT
GGTGG/
SEQ ID NO: 2
Gene N-A BIP/ AGACGGCATCATATGGGTTGCACGGGTGCCAATGTG
ATCT/
SEQ ID NO: 3
Gene N-A F3/ TGGCTACTACCGAAGAGCT/
SEQ ID NO: 4
Gene N-A B3/ TGCAGCATTGTTAGCAGGAT/
SEQ ID NO: 5
Gene N-A LF/ GGACTGAGATCTTTCATTTTACCGT/
SEQ ID NO: 6
Gene N-A LB/ ACTGAGGGAGCCTTGAATACA/

Thus, there is disclosed herein a system wherein a sample of pathogen is collected on a sampling device, the sample is efficiently eluted from the sampling device by an extraction device, whereby the limited clearance between sampling device and walls of extraction device results in extrusion of the sample into a collection zone, wherein the sample can be transferred from the collection zone to an inactivation/lysis mixture and thence to an isothermal amplification mixture with no other steps, and the amplification mixture is heated until color development indicates presence of nucleic acid sequence of original pathogen.

It will be appreciated by those skilled in the art that there are many possible modifications to be made to the specific forms of the features and components of the disclosed embodiments while keeping within the spirit of the concepts disclosed herein. Accordingly, no limitations to the specific forms of the embodiments disclosed herein should be read into the claims unless expressly recited in the claims. Although a few embodiments have been described in detail above, other modifications are possible. Other steps may be provided, or steps may be eliminated, from the described flows, and other components may be added to, or removed from, the described systems. Other embodiments may be within the scope of the following claims.

The foregoing disclosure of specific embodiments is intended to be illustrative of the broad concepts comprehended by the invention.

1. An extraction device for extracting biomaterial previously deposited on the surface of a capture element of a sampling device, the capture element being of a select cross section and size, comprising:
an upwardly opening head being wider at a top and narrower at a central opening at a bottom;
an elongate cavity attached to the enlarged head at the bottom extending downward from the head and closed at a bottom end to define an interior space, the cavity having a cross section corresponding to the cross section of the capture element and of a slightly larger size than the size of the capture element; and
an extraction fluid in the cavity interior space,
wherein, in use, insertion of the capture element through the head into the elongate cavity extrudes the extraction fluid liquid through the interface between the head and the elongate cavity and provides a fluidic shearing force that serves to solubilize biomaterial from the surface of the capture element.

2. The extraction device according to claim 1 wherein the head comprises a bowl-shaped head.

3. The extraction device according to claim 1 wherein the capture element is in the form of a flat strip.

4. The extraction device according to claim 1 wherein the capture element is cylindrical.

5. The extraction device according to claim 1 wherein clearance between an internal surface of the cavity and the surface of the capture element is less than about 1 mm.

6. The extraction device according to claim 1 wherein clearance between an internal surface of the cavity and the surface of the capture element is less than 0.1 mm.

7. The extraction device according to claim 1 wherein the extraction device is formed of injection molded plastic.

8. The extraction device according to claim 7 wherein the head and cavity are separate parts which are joined by ultrasonic welding.

9. The extraction device according to claim 7 wherein the head and cavity are separate parts which are joined by an adhesive.

10. The extraction device according to claim 1 wherein the extraction device is fabricated by blow-molding.

11. The extraction device according to claim 1 wherein the extraction device is fabricated by dip-molding.

12. A method for extracting biomaterial previously deposited on the surface of a capture element of a sampling device, the capture element being of a select cross section and size, comprising:
providing an extraction device comprising an upwardly opening head being wider at a top and narrower at a central opening at a bottom, and an elongate cavity attached to the enlarged head at the bottom extending downward from the head and closed at a bottom end to define an interior space, the cavity having a cross section corresponding to the cross section of the capture element and of a slightly larger size than the size of the capture element;
filling the cavity interior space with an extraction fluid;
inserting the capture element through the head into the elongate cavity thereby extruding the extraction fluid liquid through the interface between the head and the elongate cavity and thereby providing a fluidic shearing force that serves to solubilize biomaterial from the surface of the capture element.

13. The method according to claim 12 wherein the head comprises a bowl-shaped head.

14. The method according to claim 12 further comprising extracting a sample of the biomaterial solubilized in the extraction fluid from the extraction device.

15. The method according to claim 14 further comprising adding the sample to an inactivation reagent and LAMP mix and detergent to define a mixture.

16. The method according to claim 15 further comprising heating the mixture for a select time and determining the resultant color.

17. The method according to claim 12 wherein clearance between an internal surface of the cavity and the surface of the capture element is less than about 1 mm.

18. The method according to claim 12 wherein clearance between an internal surface of the cavity and the surface of the capture element is less than 0.1 mm.

19. The method according to claim 12 further comprising providing a cap removably received on the head to capture the extraction fluid in the extraction device.

20. The method according to claim 12 further comprising providing a cap removably, threadably received on the head to capture the extraction fluid in the extraction device.
